(19) United States
(12) Patent Application Publication
(10) Pub. No.: US 2013/0226080 A1
(43) Pub. Date: Aug. 29, 2013

(54) MEDICATED MODULE FOR A DRUG DELIVERY DEVICE

(71) Applicants: James Alexander Davies, Warwickshire (GB); Steven Wimpenny, Warwickshire (GB); Daniel Thomas De Sausmarez Lintell, Warwickshire (GB)

(72) Inventors: James Alexander Davies, Warwickshire (GB); Steven Wimpenny, Warwickshire (GB); Daniel Thomas De Sausmarez Lintell, Warwickshire (GB)

(73) Assignee: Sanofi-Aventis Deutschland GmbH, Frankfurt am Main (DE)

(21) Appl. No.: 13/881,803

(22) PCT Filed: Oct. 31, 2011
(86) PCT No.: PCT/EP2011/069101

§ 371 Date: Apr. 26, 2013

(30) Foreign Application Priority Data
Nov. 3, 2010 (EP) 10189786.6

(51) Int. Cl. A61M 5/315; A61M 5/32

(52) U.S. Cl. CPC A61M 5/31596; A61M 5/3298
USPC 604/89; 604/173

(57) ABSTRACT

A method and system for dispensing a non-user settable dose of one medicament and a user-settable dose of a primary medicament. The system may include a medicated module attachable to a drug delivery device, the drug delivery device having a drug reservoir holding a first medicament. The medicated module includes a first needle, a second needle, a collapsible feature holding a second medicament, a biasing feature operably connected to the collapsible feature, and a valve system having a closed position and an open position. During dispense, the valve system transitions from the closed position to the open position, and the biasing feature compresses the collapsible feature to force the second medicament to flow from the collapsible feature to the second needle.

### CROSS REFERENCE TO RELATED APPLICATIONS

The present application is a U.S. National Phase Application pursuant to 35 U.S.C. §371 of International Application No. PCT/EP2011/069101 filed Oct. 31, 2011, which claims priority to European Patent Application No. 10189786.6 filed Nov. 3, 2010. The entire disclosure contents of these applications are herewith incorporated by reference into the present application.

### FIELD OF DISCLOSURE

This present patent application relates to medical devices and methods of delivering at least two drug agents from separate reservoirs using devices having only a single dose setting mechanism and a single dispense interface. A single delivery procedure initiated by the user causes a non-user settable dose of a second drug agent and a variable set dose of a first drug agent to be delivered to the patient. The drug agents may be available in two or more reservoirs, containers or packages, each containing independent (single drug compound) or pre-mixed (co-formulated multiple drug compounds) drug agents. Specifically, this application concerns a medicated module having a collapsible feature holding a secondary medicament and a valve system.

### BACKGROUND

Certain disease states require treatment using one or more different medicaments. Some drug compounds need to be delivered in a specific relationship with each other in order to deliver the optimum therapeutic dose. The presently proposed devices and methods are of particular benefit where combination therapy is desirable, but not possible in a single formulation for reasons such as, but not limited to, stability, compromised therapeutic performance and toxicology.

For example, in some cases it might be beneficial to treat a diabetic with a long acting insulin and with a glucagon-like peptide-1 (GLP-1), which is derived from the transcription product of the proglucagon gene. GLP-1 is found in the body and is secreted by the intestinal L cell as a gut hormone. GLP-1 possesses several physiological properties that make it (and its analogs) a subject of intensive investigation as a potential treatment of diabetes mellitus.

There are a number of potential problems when delivering two active medicaments or “agents” simultaneously. The two active agents may interact with each other during the long-term, shelf life storage of the formulation. Therefore, it is advantageous to store the active components separately and combine them at the point of delivery, e.g. injection, needle-less injection, pumps, or inhalation. However, the process for combining the two agents needs to be simple and convenient for the user to perform reliably, repeatedly and safely.

A further problem is that the quantities and/or proportions of each active agent making up the combination therapy may need to be varied for each user or at different stages of their therapy. For example one or more active agents may require a titration period to gradually introduce a patient up to a “maintenance” dose. A further example would be if one active agent requires a non-adjustable fixed dose while the other is varied in response to a patient's symptoms or physical condition. This problem means that pre-mixed formulations of multiple active agents may not be suitable as these pre-mixed formulations would have a fixed ratio of the active components, which could not be varied by the healthcare professional or user.

Additional problems arise where a multi-drug compound therapy is required, because many users cannot cope with having to use more that one drug delivery system or make the necessary accurate calculation of the required dose combination. This is especially true for users with dexterity or computational difficulties. Further, for some drug combinations for which this delivery of two medicaments in a single injection step is desirable, it may be additionally desirable for the two medicaments to be delivered substantially sequentially (i.e., one after the other, with minimal or no opportunity for mixing. Avoidance of mixing of the 2 drug formulations might have several advantages. For example, it is known that the pharmacokinetics of certain drugs are critically dependent on their concentration. By delivering two drugs sequentially with no mixing the optimal concentration of each drug for optimal pharmacokinetics can be maintained. In addition, certain drugs have to be formulated in particular solvent environments (eg a a specific pH range) to remain in solution. By delivering two drugs sequentially with no mixing the optimal pH range and therefore solubility can be maintained during delivery. Additional issues arrive due to the ullage volume left in a drug delivery device post dispense. For instance, a large ullage volume may result in a large volume of “wasted” medicament.

Accordingly, there exists a need to provide devices and methods for the delivery of two or more medicaments in a single injection or delivery step that is simple for the user to perform and that limits or minimizes the ullage volume left in the drug delivery device post dispense. The presently proposed devices and methods overcome the above-mentioned problems by providing separate storage containers for two or more active drug agents and, specifically, a collapsible feature that holds the second medicament. Beneficially, delivering the drugs sequentially may avoid or limit mixing of the drug agents. A user sets a dose of one medicament (i.e., a user settable dose). The dose of the second medicament is independently controlled and therefore is not influenced by an amount of the user settable dose (i.e., non-user settable). The drug agents are then delivered to the patient during a single delivery procedure. Beneficially, the collapsible container holding the second medicament is compressed during drug delivery, and this compression serves to reduce or minimize the ullage volume left in the medicated module post dispense.

The proposed devices and methods also give the opportunity for varying the quantity of one or both medicaments. For example, one fluid quantity can be varied by changing the properties of the injection device (e.g., dialing a user variable dose or changing the device's “fixed” dose). The second fluid quantity can be changed by manufacturing a variety of secondary drug containing packages with each variant containing a different volume and/or concentration of the second active agent. The user or healthcare professional would then select the most appropriate secondary package or series or combination of series of different packages for a particular treatment regime. The proposed medicated module forms a self-contained reservoir in which non-user-settable dose of a medicament may be stored.

These and other advantages will become evident from the following more detailed description of the invention.

### SUMMARY

The presently proposed devices and methods allow for complex combinations of multiple drug compounds within a single drug delivery system. Further, in given embodiments, the presently proposed devices and methods minimize ullage and allow the user to substantially sequentially dispense at least two drug agents through one single dose setting mechanism and a single dispense interface. This single dose setter controls the mechanism of the device such that a predefined combination of the individual drug compounds is delivered when a single dose of one of the medicaments is set and dispensed through the single dispense interface.

By defining the therapeutic relationship between the individual drug compounds, the proposed delivery device and delivery methods help ensure that a patient/user receives the optimum therapeutic combination dose from a multi-drug compound device without the inherent risks associated with multiple inputs where the user has to calculate and set the correct dose combination every time they use the device. The medicaments can be fluids, defined herein as liquids or gases or powders that are capable of flowing and that change shape at a steady rate when acted upon by a force tending to change its shape.

Applicants' proposed concept is of particular benefit to users with dexterity or computational difficulties as the single input and associated predefined therapeutic profile removes the need for them to calculate their prescribed dose every time they use the device and the single input allows considerably easier setting and dispensing of the combined compounds.

In a preferred embodiment a master drug compound, such as insulin, contained within a multiple dose, user selectable drug delivery device could be used with a single use, user replaceable, medicated module that contains a single dose of a secondary medicament and the single dispense interface. When connected to the primary drug delivery device, the secondary compound is activated/delivered on dispense of the primary compound. Although the present application specifically mentions insulin, insulin analogs or insulin derivatives, and GLP-1 or GLP-1 analogs as two possible drug combinations, other drugs or drug combinations, such as an analgesics, hormones, beta agonists or corticosteroids, or a combination of any of the above-mentioned drugs could be used with our proposed method and system.

For the purposes of our proposed method and system the term “insulin” shall mean Insulin, insulin analogs, insulin derivatives or mixtures thereof, including human insulin or a human insulin analogs or derivatives. Examples of insulin analogs are, without limitation, Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin or Des(B30) human insulin. Examples of insulin derivatives are, without limitation, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl- ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta-decanoyl) human insulin.

As used herein the term “GLP-1” shall mean GLP-1, GLP-1 analogs, or mixtures thereof, including without limitation, exenatide (Exendin-4(1-39), a peptide of the sequence H- His-Gly-Glu-Gly-Thr-Phe-Thr-Ser-Asp-Leu-Ser-Lys-Gln-Met-Glu-Glu-Glu-Ala-Val-Arg-Leu- Phe-Ile-Glu-Trp-Leu-Lys-Asn-Gly-Gly-Pro-Ser-Ser-Gly-Ala-Pro-Pro-Pro-Ser-NH2), Exendin-3, Liraglutide, or AVE0010 (H-His-Gly-Glu-Gly-Thr-Phe-Thr-Ser-Asp-Leu-Ser-Lys-Gln-Met-Glu- Glu-Glu-Ala-Val-Arg-Leu-Phe-Ile-Glu-Trp-Leu-Lys-Asn-Gly-Gly-Pro-Ser-Ser-Gly-Ala-Pro- Pro-Ser-Lys-Lys-Lys-Lys-Lys-Lys-NH2).

Examples of beta agonists are, without limitation, salbutamol, levosalbutamol, terbutaline, pirbuterol, procaterol, metaproterenol, fenoterol, bitolterol mesylate, salmeterol, formoterol, bambuterol, clenbuterol, indacaterol.

Hormones are for example hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

According to an embodiment, a medicated module is attachable to a drug delivery device holding a first medicament. The medicated module includes a first needle, a second needle, a collapsible feature holding a second medicament, a biasing feature operably connected to the collapsible feature, and a valve system comprising a flow channel. The valve system has a closed position and an open position. Further, the valve system is in fluid communication with the first needle and the collapsible feature. Still further, the flow channel is in fluid communication with the second needle. When the valve system is in the closed position, the valve system prevents fluid communication between (i) the second needle and the collapsible feature and (ii) the second needle and the first needle. During dispense, the valve system transitions from the closed position to the open position. When the valve system is in the open position, (i) the flow channel is in fluid communication with the collapsible feature and the first needle, and (ii) the biasing feature compresses the collapsible feature, wherein the compression forces the second medicament to flow from the collapsible feature to the second needle.

According to another embodiment, a drug delivery system to deliver a non-user settable dose of one medicament followed by a user-settable dose of a primary medicament through a single dose setter and a single dispense interface is provided. The drug delivery system includes a drug delivery device that comprises a housing including a single dose setter operably connected to a primary reservoir of medicament including the primary medicament. The drug delivery device also comprises a dose button operably connected to the primary reservoir of medicament. The drug delivery system also includes a medicated module attached to the drug delivery device. The medicated module comprises a first needle in fluid communication with the primary reservior, a second needle, a collapsible feature holding a second medicament, a biasing feature operably connected to the collapsible feature, and a valve system comprising a flow channel. The valve system has a closed position and an open position. Further, the valve system is in fluid communication with the first needle and the collapsible feature. Still further, the flow channel is a flow channel in fluid communication with the second needle.

When the valve system is in the closed position, the valve system prevents fluid communication between (i) the second needle and the collapsible feature and (ii) the second needle and the first needle. During dispense, the valve system transitions from the closed position to the open position due to a force of the primary medicament flowing from the reservoir to the valve system. When the valve system is in the open position, (i) the flow channel is in fluid communication with the collapsible feature and the first needle, and (ii) the biasing feature compresses the collapsible feature, wherein the compression forces the second medicament to flow from the collapsible feature to the second needle. The compression may force the second medicament from the collapsible feature via the flow channel to the second needle.

A medicated module in accordance with Applicants' proposed concept can be designed for use with any drug delivery device with an appropriate compatible interface. However, it may be preferable to design the module in such a way as to limit its use to one exclusive primary drug delivery device (or family of devices) through employment of dedicated or coded features to prevent attachment of a non-appropriate medicated module to a non-matching device. In some situations it may be beneficial to ensure that the medicated module is exclusive to one drug delivery device while also permitting the attachment of a standard drug dispense interface to the device. This would allow the user to deliver a combined therapy when the module is attached, but would also allow delivery of the primary compound independently through a standard drug dispense interface in situations, such as, but not limited to, dose splitting or top-up of the primary compound.

According to the present invention a medicated module may comprise a first needle. The first needle may be configured to establish fluid communication with the primary reservoir. The first needle or engagement needle may engage with or may communicate with a reservoir of drug delivery device when the medicated module is attached to a drug delivery device. The first needle may be configured to pass through a septum of a cartridge holding a first medicament.

According to the present invention a medicated module may comprise a second needle. The second needle may be configured for subcutaneously inject medicament into an injection site. The second needle may also be referred to as an output needle.

According to the present invention a medicated module may comprise a valve system. The valve system may include a valve seal. The valve system may include a recess. The recess may be configured to allow for axial movement of the valve seal. When the valve system transitions from a closed position to an open position, the valve seal may move through the recess in distal direction.

According to the present invention a medicated module may be attachable to a drug delivery device holding a first medicament. The medicated module may comprise a valve system comprising a flow channel, the valve system having a closed and an open position. During dispense, the valve system transitions from the closed position to the open position due to a force of a first or primary medicament flowing from a reservoir of the drug delivery device to the valve system. The valve system is transitioned from the closed to the open position when the first medicament is dispensed from the drug delivery device to the valve system.

According to the present invention a medicated module may comprise a biasing feature. The biasing feature may be configured to compress the collapsible feature, wherein the compression may force the second medicament to flow out the collapsible feature. The biasing feature may facilitate dispense of the secondary medicament. The biasing feature may be preloaded and provides a compression force against the collapsible feature.

According to the present invention a medicated module holding a second medicament may comprise a biasing feature, wherein, depending on the force of the biasing feature and that exerted by a user when dispensing the first medicament, the second medicament and first medicament may be partially mixed as they are delivered. In one embodiment the medicated module may be attachable to a drug delivery device holding a first medicament and having a dose dispense button. Depending on the force of the biasing feature and that exerted by the user on the dose dispense button, the second medicament and first medicament may be partially mixed as they are delivered to the user. Adapting the force of the biasing feature may be one option to adjust the mixing ratio. Depending on the force of the biasing feature and the force exerted by a user when dispensing the first medicament, the second medicament and first medicament may be partially mixed as they are delivered. However, complete mixture is also feasible. Another option to adjust the mixing ratio may be the configuration of the valve system. For example, the valve system may comprise a flow channel adapted to prevent mixing in having a relatively small volume. Otherwise, a flow channel having a relatively large volume would be adapted to support mixing. The terms relatively small and relatively large have to be understood in the context of the fluid dynamics of the configuration of the valve system. A volume that would be adapted to allow the first medicament to flow from the reservoir of a drug delivery device to the second needle without additional friction would be the threshold. A volume smaller than that, would reduce the flow of first medicament and thus allow the second medicament to be dispensed by means of the force from the biasing feature. Hence, this kind of configuration would be beneficial to at least reduce mixing. On the other hand, a volume larger than the threshold volume, would not reduce the flow of the first medicament. A flow of the second medicament could be easily carried by the flow channel. Hence, this kind of configuration would provide for supporting mixing of the two medicaments.

A particular benefit of Applicants' methods and systems is that the methods and systems serve to limit or minimize the amount of ullage left in the medicated module post use (relative to the volume of the fixed dose of medicament in the module). In addition, in given embodiments, Applicant's method and systems allow a relatively large volume of the secondary medicament to be dispensed when only a relatively small volume of the primary medicament is selected and dispensed. In order for the valve to be opened, and the secondary medicament dispensed under the action of the biasing element, only a relatively small volume of the primary medicament needs to flow to urge the valve forward into its open state. Further, in given embodiments, Applicants' method and systems allow for sequential dosing of a first medicament and a second medicament through a single dispense interface. Thus, the methods and systems beneficially prevent or limit mixing of the first, primary medicament and the second medicament. This may be beneficial, for example, when mixing of medicaments negatively or detrimentally affects at least one of the medicaments.

In a preferred embodiment, the primary drug delivery device is used more than once and therefore is a multi-use device; however, the drug delivery device may also be a single use disposable device. Such a device may or may not have a replaceable reservoir of the primary drug compound, but Applicants' proposed concept is equally applicable to both scenarios. It is also possible to have a suite of different medicated modules for various conditions that could be prescribed as one-off extra medication to patients already using a standard drug delivery device. Should the patient attempt to reuse a previously used medicated module, features may be present that prevent reattachment to a primary drug delivery device or that prevent or discourage subsequent dosing through the needle via alternative means. For example, this module may include a locking needle guard that is activated after a user delivers a dose from the medicated module. Other means of alerting the user may include some (or all) of the following:

Physical prevention of medicated module re-attachment to the primary drug delivery device once the module has been used and removed.

Physical/hydraulic prevention of subsequent liquid flow through the drug dispense interface once it has been used.

Physical locking of the dose setter and/or dose button of the primary drug delivery device.

Visual warnings (e.g., change in color and/or warning text/indicia within an indication window on the module once insertion and/or fluid flow has occurred).

Tactile feedback (presence or absence of tactile features on the outer surface of the module hub following use).

A further proposed feature is that both medicaments are delivered via one injection needle and in one injection step. This offers a convenient benefit to the user in terms of reduced user steps compared to administering two separate injections. This convenience benefit may also result in improved compliance with the prescribed therapy, particularly for users who find injections unpleasant or who have computational or dexterity difficulties.

These as well as other advantages of various aspects of the present invention will become apparent to those of ordinary skill in the art by reading the following detailed description, with appropriate reference to the accompanying drawings.

### BRIEF DESCRIPTION OF THE DRAWINGS

Exemplary embodiments are described herein with reference to the drawings, in which:

FIG. 1 illustrates a perspective view of one possible drug delivery device that can be used with Applicants' proposed medicated module;

FIG. 2 illustrates a cross-sectional view of an exemplary medicated module attached to an exemplary drug delivery device; and

FIG. 3 illustrates a cross-sectional view of the exemplary medicated module and the exemplary drug delivery device of FIG. 2 during dispense.

### DETAILED DESCRIPTION

Applicants' proposed concept is a system and method for dispensing a non-user settable dose of one medicament and a user-settable dose of a primary medicament using a single dispense interface. The medicaments may be dispensed substantially sequentially. The proposed concept relates specifically to a method and system that utilizes a collapsible feature and a valve system that facilitate delivering of a secondary medicament from a medicated module and a primary medicament from a primary drug delivery device through the same output needle in a single injection step. Beneficially, Applicants' proposed concept serves to minimize or limit the ullage volume left in the medicated module (relative to the volume of the secondary medicament disposed from it). In addition, Applicants' proposed concept provides a means by which a relatively large volume of the secondary medicament can be dispensed even if a volume of the primary medicament smaller than this is selected and dispensed.

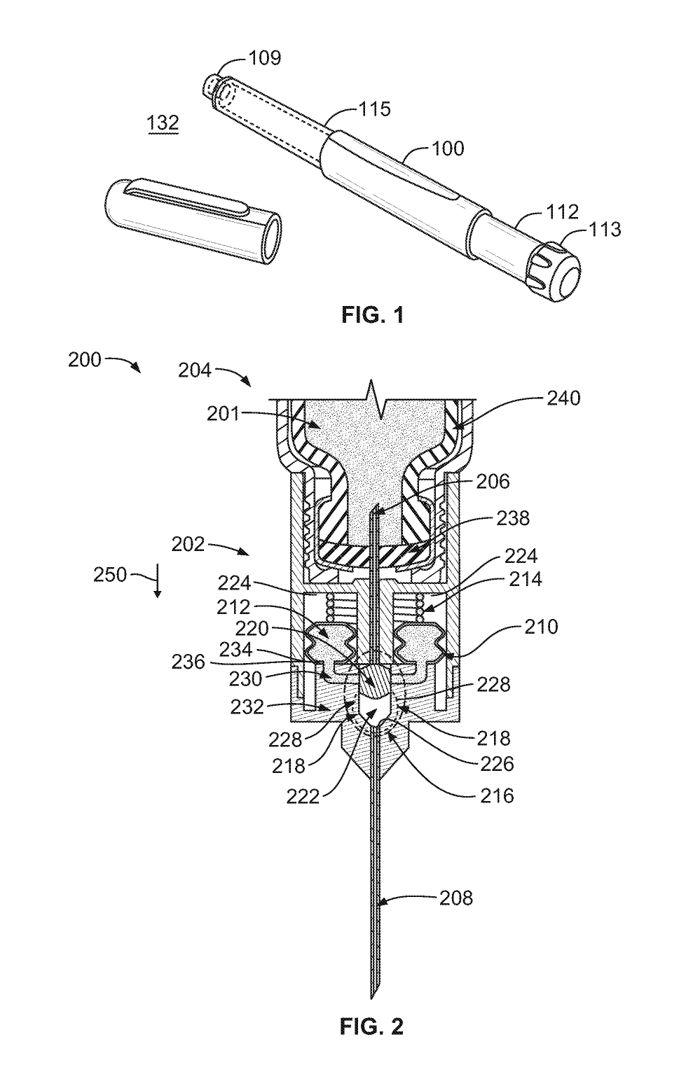

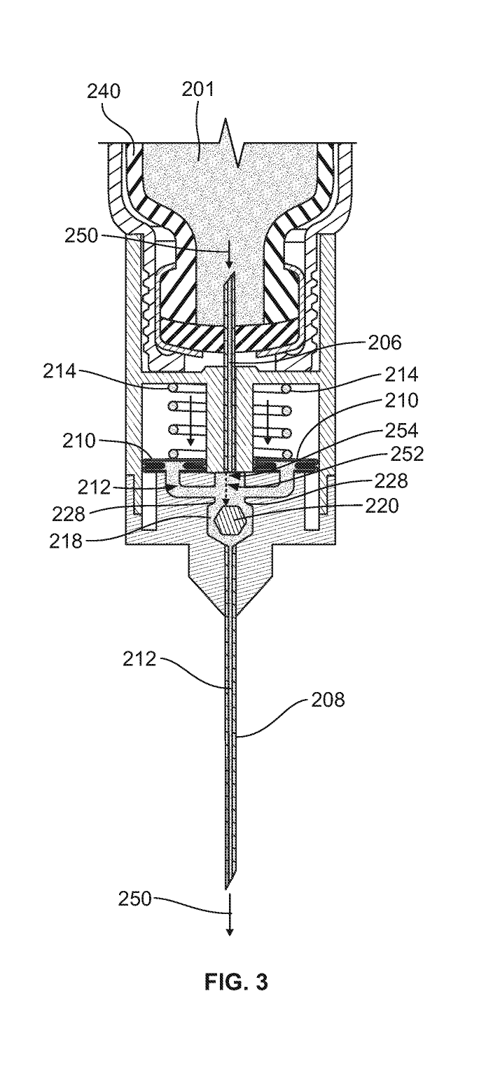

A medicated module in accordance with embodiments of Applicants' proposed concepts may be attached to a primary drug delivery device, such as drug delivery device 100. FIG. 1 illustrates one example of a drug delivery device 100 to which a medicated module, such as the exemplary medicated module depicted in FIGS. 2-3, can be attached. Specifically, the medicated module can be attached to the connection means 109 of distal end 132. A medicated module in accordance with Applicants' proposed concept is preferably self-contained and provided as a sealed and sterile disposable module that has an attachment means compatible to the attachment means 109 at the distal end 132 of device 100. Although not shown, the medicated module could be supplied by a manufacturer contained in a protective and sterile container, where the user would peel or rip open a seal or the container itself to gain access to the sterile medicated module. Further, the drug delivery device 100 includes a housing including a single dose setter 112. The dose setter 112 may be operably connected to a primary reservoir of medicament that may be stored in the drug delivery device, such as in cartridge holder 115. The user may use a dose dial button 113 in order to dial a user selectable dose of the primary medicament.

Applicants' proposed concept is a medicated module that is attachable to a drug delivery device (such as drug delivery device 100) that has a drug reservoir holding a first, primary medicament. The proposed medicated modules include a collapsible feature holding a secondary medicament and valve system. Beneficially, the medicated modules operate in such a way that the ullage in the medicated module after dispense is minimized relative to the volume of medicament originally in the medicated module.

An exemplary medicated module is now described with reference to FIGS. 2 and 3. Specifically, FIG. 2 illustrates a drug delivery system 200 including medicated module 202 and primary drug delivery device 204 that contains a first, primary medicament 201. In FIG. 2, only a partial view of the distal end of the primary drug delivery device 204 is shown. Primary drug delivery device 204 may be the same as or similar to drug delivery device 100 of FIG. 1. The medicated module 202 includes a first needle 206 and a second needle 208. The first needle 206 may be referred to herein as an “engagement needle”, as the needle engages with or communicates with the reservoir 240 of drug delivery device 204 when medicated module 202 and device 204 are attached. Further, the second needle 208 may be referred to herein as an “output needle”, as the second needle may be used to subcutaneously inject medicament into an injection site, such as an injection site of a user of drug delivery system 200.

The medicated module 202 also includes a collapsible feature 210 that holds a second medicament 212. The collapsible feature may be composed of any suitable material capable of securely holding a medicament and collapsing or compressing under a pre-determined force. Preferably, this suitable material may be an inert material when placed into long term contact with second medicament and that offers good performance with respect to leachables and/or extractables. For example, the collapsible feature may be composed of; TPE (Thermoplastic Elastomer), Liquid Silicone Rubber (LSR) and natural rubbers. Alternative materials, including Low-density Polyethylene (LDPE) or Linear low-density Polyethylene (LLDPE) are also possible. Where improved barrier properties are desirable, laminate materials may be used e.g. multilayer materials consisting of the primary membrane material (potentially as above) plus additional thin layers of materials like PVC (Polyvinyl chloride) PCTFE (Polychlorotrifluoro ethylene) or Aluminum. Other materials and/or materials combinations are also possible. The medicated module 202 also includes a biasing feature 214 that is operably connected to the collapsible feature 210. In an example, the biasing feature or element 214 is a spring. The biasing feature facilitates dispense of the secondary medicament 212, as described in more detail below. Further, the biasing element 214 may be operably connected to a proximal internal surface of the medicated module, such as surface 224. The biasing element 214 is a preloaded spring that pressurizes the collapsible feature 210. The preload may either be provided at the point of manufacture of the medicated module or may, preferably, be generated as a direct outcome of the user attaching the medicated module to the primary device (e.g., the biasing element could be in a substantially relaxed condition when manufactured but when the medicated module is attached to the primary device the axial engagement of the two elements could be utilized to compress the biasing element to it's biased condition). This may be beneficial as it means that the contents of the collapsible feature are not held in a pressurized state for an extended period of time.

In an example, the second medicament 212 located in the collapsible feature 210 comprises a GLP-1 or GLP-1 l analog and the first medicament 201 located in the primary drug delivery device 204 comprises an insulin or an insulin analog. Other examples of medicaments and combinations of medicaments are possible as well.

The medicated module also includes a valve system 216 that is capable of both an open position and closed position. The valve system 216 may encompass all of the elements shown in the dashed-oval shape in FIG. 2. Generally, FIG. 2 shows valve system 216 in the closed position, and FIG. 3 shows valve system 216 in the open position. The valve system includes a first flow path 218, which is a flow path in fluid communication with the output needle 208. Further, the valve system includes a valve seal 220 and a recess 222. The recess is configured to allow for axial movement of the valve seal. In particular, when the valve system 216 transitions from a closed position to an open position, the valve seal 220 moves through the recess in distal direction 250.

Flow path 218 is preferably a path that travels around the valve seal 220 through recess 222 to an opening 226 of the output needle 208. Specifically, the flow path 218 may have an axial portion that flows in distal direction 250 and a radial portion that flows to the opening 226 of the output needle 208. Recess 222 may include at least one aperture 228 that serves as an opening to the flow path 218. Flow path 218 may also be referred to herein as a “diverting channel” or “flow channel,” as the flow path 218 serves to divert medicament around the valve seal 220 after the seal 220 traverses past the opening 228 during the transition from a closed position to an open position. The operation of the valve system 216 will be described in more detail below in reference to the drug dispense process.

The medicated module 202 may also include a flow path from the collapsible feature 210 to the valve system 216, such as second flow path 230. Flow path 230 is a path that travels through internal body portion 232. In this example, as shown in FIGS. 2 and 3, the distal end 234 of the collapsible feature 210 rests on the proximal surface 236 of the internal body feature 232.

FIG. 2 depicts the medicated module 202 after the module 202 is attached to drug delivery device 204 and prior to the dispense process. Attachment of medicated module 202 to drug delivery device 204 causes the engagement needle 206 to penetrate the septum 238 of the drug cartridge or reservoir 240 of the drug delivery device 204. Once the engagement needle 206 has passed through the septum 238 of the cartridge 240, fluid connection is made with the first, primary medicament 201. In other words, the first needle 206 is in fluid communication with the drug reservoir 240. At this stage, the valve system 216 will prevent the first medicament 201 from advancing beyond the valve seal 220.

As can be seen in FIG. 2, in the closed position, the valve system 216 prevents fluid communication between the diverting channel 218 and the collapsible member 210. The valve system 216 also prevents fluid communication between the diverting channel 218 and the engagement needle 206. As such, the valve system 216 blocks the collapsible feature 210 and the engagement needle 206 from communicating with the output needle. Therefore, at this stage, neither the second medicament 212 nor the first medicament 201 can reach the output needle 208 and, therefore, the medicaments 201, 212 cannot yet be injected.

As shown in FIG. 2, in the closed position, the valve seal 220 is located against a radial portion of the second flow path 230. Therefore, although the biasing feature 214 is a preloaded spring element that provides a compression force against the collapsible feature 210, the collapsible feature 210 does not collapse under the force of the preloaded spring. The feature 210 does not collapse because the second medicament 212 provides a radial force against the valve seal 220 that is perpendicular to its axis of operation, and this radial force therefore cannot move the valve system 216 from its closed position to its open position. The volume of the second medicament 212 in the collapsible feature 210 prevents the collapsible feature from being compressed under the force of the preloaded spring element 214.

In this example depicted in FIGS. 2 and 3, a force acting along the axis of operation for the valve (i.e., in direction 250) is required to move the valve system 216 from an closed position to an open position. Since the valve seal 220 is located below the engagement needle 206, primary medicament that flows from the engagement needle to the valve system 216 may provide such an axially-directed force against the proximal end of the valve seal 220. As will be described in more detail below, this axially-directed force may cause the valve system to transition to its open position when a user begins the dispense process.

It should be understood, however, that after connection and prior to dispense by the user, a small amount of medicament 201 may flow through the needle 206 to the valve system 216. However, at this stage, the valve system preferably will remain in the closed position. The valve system 216 may be designed such that a predetermined force and/or volume is required to move the valve system 216 to the open position, and the force and/or volume of any medicament 201 that flows through needle 206 to the valve system prior to any dispense action by the user will preferably be insufficient to move the valve system 216 into the open position. An example force required to move the valve seal is approximately 8-12 Newtons applied to the Dose Dial Button which may equate to approximately 1-1.5 Newtons on the valve seal. An exemplary volume required for full axial displacement of the valve seal may comprise approximately 10-15 mm3 Preferably, in order to provide such a force via the primary medicament 201, a user initiates the dispense process, as described below.

After the module 202 is attached to the device 204, a user may set a user-settable dose of the first medicament 201. The dose of the drug delivery device may be set in a usual manner (e.g., by dialing out an appropriate number of units of the primary medicament 201 with a dose dial of drug delivery device 204). Dispense of the second medicament 222 and the first medicament 201 may then be achieved via activation of the dosing mechanism of the drug delivery device.

As the primary medicament 201 is first dispensed from the reservoir 240 at the beginning of the dispense process, the medicament 201 flows through the engagement needle 206 in distal direction 250 into a cavity 252 formed between the valve seal 220 and the end 254 of the engagement needle 206. As the main body of the medicated module 202 is substantially rigid and the medicament 201 is effectively incompressible, the valve seal 220 is displaced axially as the liquid 201 is dispensed into the cavity 252.

Displacement of the valve seal 220 causes the valve system 216 to open fluid communication between the second medicament 212 and the output needle 208 once the proximal edge of the valve seal 220 traverses past the proximal edge of the diverting channel 218 of the valve system 216. Specifically, when the valve seal traverses past the opening 228 of the diverting channel 218, the preloaded spring 214 begins to compress the collapsible feature 210. The force of the preloaded spring 214 causes the collapsible feature to compress in direction 250. This compression forces the second medicament to flow out of the collapsible feature 210, through flow channel 230, then through diverting channel 218, and finally through output needle 208. Thus, as shown in FIG. 3, the preloaded spring 214 forces substantially all of the second medicament 212 to be dispensed from output needle 208. The medicament 212 being fully dispensed preferably corresponds to when the collapsible feature 210 is substantially fully compressed.

The first medicament 201 may then be dispensed from the medicated module as the user completes the dispense stroke. The medicament 201 is forced from the reservoir 240 through engagement needle 208, then through diverting channel 218, and finally through output needle 208. After the user finishes dispensing the first medicament 201, the user may remove the output needle 208 from the injection site. Then, the depleted medicated module 202 may be disposed of. Assuming that the drug delivery device 204 still holds some first medicament 201, the drug delivery device 204 may be reused by the patient as required.

After the second medicament 212 has been fully dispensed, the collapsible feature 210 preferably has a minimal internal volume, as shown in FIG. 3. Additionally, the presence of the spring force acting on the collapsible feature 210 ensures that the collapsible feature 210 does not backfill with the primary medicament 201 during the rest of the dispense stroke. In the disclosed embodiments, additional features may be present on the collapsible feature 210 and/or the body of the medicated module to ensure that, once compressed, the collapsible feature does not return to its original, non-collapsed form. Such additional features could include standard one-way clip or ratchet type arrangements configured to operate between the collapsible feature and/or part of the housing or biasing element. Additionally, the collapsible feature could be configured to be a bi-stable feature, that is, once a certain amount of the secondary medicament has been dispensed under the action of the biasing element, the form of the collapsible bi-stable feature ‘snaps’ to a biased closed arrangement. Further arrangements to prevent back flow of the primary medicament into the collapsible feature during dispense are also possible.

Preferably, as shown in FIGS. 2 and 3, the collapsible feature is a ring-shaped feature. The ring shaped feature may be disposed in the body around the engagement needle 206. However, the collapsible feature of Applicants' proposed concept may be any other suitable design.

Because the collapsible feature 210 post dispense has a volume less than the original volume, the ullage that may be present in the medicated module 202 after dispense is reduced. In some situations, it may be preferable for the ullage volume in the medicated module (post dispense) to be smaller than the volume of the fixed-dose medicament (i.e., the second medicament) dispensed by it. This is of particular benefit where the volume of the fixed-dose medicament needs to be relatively large relative to the potential dose from the primary drug delivery device (e.g., greater than 0.05 ml in the example of pen-type injection devices for the delivery of insulin).

Beneficially, limiting the ullage in the medicated module may help reduce the amount of medicament that is wasted by being left in the medicated module after use. Further, limiting the ullage in the medicated module may help reduce the degree to which the dialed dose (i.e., what is indicated to the patient on the dose setter) on the primary delivery device needs to be offset to accommodate for the effect of ullage in the single-use medicated module.

Additionally, the described arrangement is such that the two medicaments are likely to be delivered generally sequentially, provided that the force exerted by the biasing feature is high enough. For example, if the preloaded biasing element 214 provides a force that is greater than the force provided by the user during dispense, the spring will likely force the second medicament 212 to expel from output needle 208 before the dispense stroke of the user forces the first medicament 201 to output needle 208. As a particular example, the preloaded spring may provide a force of approximately 15 to approximately 20 Newtons. Therefore, substantially all of the second medicament may be dispensed from the module before the dose of the first medicament 201 is dispensed.

Thus, beneficially, the proposed concept prevents or limits the mixing of the two medicaments within the device. This could be beneficial for applications whereby the efficacy of the combined medication reduces over time and so it is best for the mixing to occur as close to in vivo as possible to maximum the therapeutic effect. However, it should be understood that, depending on the force of the biasing feature 214 and that exerted by the user on the Dose Dispense Button, the second medicament 212 and first medicament 201 may be partially mixed as they are delivered to the user. Additionally, since some of the first medicament forces open the valve seal 220 before the second medicament is dispensed, a small amount of the first medicament 201 will likely be dispensed along with the second medicament 212.

In an alternative embodiment, attachment of the medicated module to the drug delivery device preloads the spring element that acts on the collapsible feature. Preloading the spring element in this fashion may be useful as it means that the collapsible feature is in its pressurized condition after the medicated module has been attached to the drug delivery by the user. One advantage of such an arrangement is that there would be reduced risk of leakage of medicament, either directly via the valve, or through accelerated permeability through the collapsible membrane materials. Further, such an arrangement would also tend to reduce the risk of creep in the medicated module, particularly with respect to medicated modules comprising certain plastic elements.

Such preloading upon attachment may be achieved by, for example, an axially moveable lower portion of the medicated module that compresses the spring element during attachment. A retention feature may also be used to prevent the moveable lower portion of the medicated module from returning to its pre-attached position. Other examples of preloading the biasing member upon attachment are possible as well.

The connection or attachment between the medicated module of the above described embodiments may contain additional features (not shown), such as connectors, stops, splines, ribs, grooves, and the like design features, that ensure that specific medicated module are attachable only to matching drug delivery devices. Such additional features would prevent the insertion of a non-appropriate medicated module to a non-matching injection device.

The shape of the medicated module may be a cylindrical body or any other geometric shape suitable for defining a fluid reservoir or for containing discrete self-contained reservoir of the medicament in the medicated module and for attaching one or more needle cannula. The medicated module may be manufactured from glass or other drug contact suitable material. The integrated output needle can be any needle cannula suitable for subcutaneous or intramuscular injection. Preferably the medicated module is provided by a drug manufacturer as a stand-alone and separate device that is sealed to preserve sterility. The sterile seal of the module is preferably designed to be opened automatically, e.g. by cutting, tearing or peeling, when the medicated module is advanced or attached to the drug delivery device by the user.

The medicated module of Applicants' concept should be designed to operate in conjunction with a multiple use injection device, preferably a pen-type multi-dose injection device, similar to what is illustrated in FIG. 1. The injection device could be a reusable or disposable device. By disposable device it is meant an injection device that is obtained from the manufacturer preloaded with medicament and cannot be reloaded with new medicament after the initial medicament is exhausted. The device may be a fixed dose or a settable dose and preferably a multi-dose device, however, in some cases it may be beneficial to use a single dose, disposable device.

A typical drug delivery device contains a cartridge or other reservoir of medication. This cartridge is typically cylindrical in shape and is usually manufactured in glass. The cartridge is sealed at one end with a rubber bung and at the other end by a rubber septum. The drug delivery pen is designed to deliver multiple injections. The delivery mechanism is typically powered by a manual action of the user, however, the injection mechanism may also be powered by other means such as a spring, compressed gas or electrical energy.

Exemplary embodiments of the present invention have been described. Those skilled in the art will understand, however, that changes and modifications may be made to these embodiments without departing from the true scope and spirit of the present invention, which is defined by the claims.

1-14. (canceled)

15. A medicated module attachable to a drug delivery device, the drug delivery device having a drug reservoir holding a first medicament, the medicated module comprising:
a first needle;
a second needle;
a collapsible feature, wherein the collapsible feature holds a second medicament;
a biasing feature operably connected to the collapsible feature; and
a valve system comprising a flow channel, wherein the valve system has a closed position and an open position, wherein the valve system is in fluid communication with the first needle, wherein the valve system is in fluid communication with the collapsible feature, and wherein the flow channel is in fluid communication with the second needle,
wherein, when the valve system is in the closed position, the valve system prevents fluid communication between (i) the second needle and the collapsible feature and (ii) the second needle and the first needle,
wherein, during dispense the valve system transitions from the closed position to the open position, and
wherein, when the valve system is in the open position, (i) the flow channel is in fluid communication with the collapsible feature and the first needle, and (ii) the biasing feature compresses the collapsible feature, wherein the compression forces the second medicament to flow from the collapsible feature to the second needle.

16. The medicated module of claim 15, wherein, during dispense, a dose of the first medicament is forced to flow from the reservoir through the valve system to the second needle.

17. The medicated module of claim 16, wherein the dose of the first medicament is forced to flow to the second needle after substantially all of the second medicament is forced to flow from the collapsible feature to the second needle.

18. The medicated module of claim 15, wherein, during dispense, the first medicament flows through the first needle to the valve system, and wherein the first medicament forces the valve system to transition from the closed position to the open position.

19. The medicated module of claim 18, wherein a force required to move the valve system from a closed position to an open position is in a range of approximately 8 Newtons to approximately 12 Newtons.

20. The medicated module of claim 15, wherein the valve system further comprises a valve seal and a recess, and when the valve system transitions from the closed position to the open position, the valve seal moves in a distal direction through the recess.

21. The medicated module of claim 20, wherein when the valve system transitions from the closed position to the open position, the valve seal traverses past an opening to the flow channel.

22. The medicated module of claim 20, wherein, in the open position, the valve seal is located in a distal end of the recess.

23. The medicated module of claim 20, wherein the valve seal prevents the biasing feature from compressing the collapsible feature when the valve system is in the closed position.

24. The medicated module of claim 15, wherein the biasing feature is loaded upon attachment of the medicated module to the drug delivery device.

25. The medicated module of claim 24, wherein the medicated module further comprises an internal surface that compresses the biasing feature during attachment of the medicated module to the drug delivery device.

26. The medicated module of claim 24, wherein the biasing feature comprises a spring.

27. The medicated module of claim 15, wherein the collapsible feature is a ring-shaped feature.

28. The medicated module of claim 15, wherein depending on the force of the biasing feature and that exerted by the user when dispensing the first medicament, the second medicament and first medicament may be partially mixed as they are delivered.
